(19) United States
(12) Patent Application Publication
(10) Pub. No.: US 2010/0049096 A1
(43) Pub. Date: Feb. 25, 2010

(54) SYSTEM FOR FALL PREVENTION AND A METHOD FOR FALL PREVENTION USING SUCH A SYSTEM

(75) Inventor: Warner Rudolph Theophile Ten Kate, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/513,508

(22) PCT Filed: Nov. 9, 2007
(86) PCT No.: PCT/IB07/54560

§ 371 Date: May 5, 2009

(30) Foreign Application Priority Data
Nov. 14, 2006 (EP) 06124031.3

(51) Int. Cl. A61B 5/103

(52) U.S. Cl. 600/595; 600/587

(57) ABSTRACT

System for fall prevention for a user, comprising a number of sensors (2) attachable to at least one lower body segment (3), wherein said sensors (2) are adapted to measure movement of said at least one lower body segment (3) and to translate the movement into a signal (S), the system further comprising a control (12) adapted to receive the signal (S) from said respective sensors (2), wherein in use the control (12) observes the signal (S) as an actual sequence of postures of said at least one lower body segment (3) and compares the actual sequence with a predetermined sequence of postures as a function of time, (the predetermined sequence relating to a low risk of falling,) wherein the control (12) is adapted to determine a high risk of falling when the actual sequence deviates from the predetermined sequence (to a certain degree). The invention further relates to a method for fall prevention using such a system for fall prevention.

### FIELD OF THE INVENTION

The invention relates to a system for fall prevention for a user.

### BACKGROUND OF THE INVENTION

For fall prevention, more specific fall detection, it is known for a user to wear an accelerometer, for instance worn in a housing connected to the belt of the user. The accelerometer triggers on high impact and/or free-fall acceleration. Additional parameters for refining the triggering could be detecting horizontal position and duration of staying in said position after an incident. After an incident, like falling, occurs, the accelerometer can warn a service centre, which calls back the user over a telephone line and subsequently decides about actions to take in order to help a user.

Furthermore, other systems for fall detection are known. For instance a user can be supplied with an emergency button, usually worn at a cord around the neck of the user. In case of an accident, for instance falling down, the user can press the emergency button to warn a service centre that is connected to the emergency button or somebody else. A disadvantage of these systems is that they lack full reliability. Furthermore, they do not actually prevent for falling but warn in case a user already has fallen. However, users that are insecure during walking, for example caused or enhanced by a fear of falling or by fatigue in the muscles, are helped with a system for fall prevention, which decreases the actual risk of falling or at least helps them to avoid situations of a higher risk of falling and feeling more safe.

### SUMMARY OF THE INVENTION

It is therefore an object of the invention to provide a system for fall prevention of the abovementioned type, wherein the disadvantages of the known systems are minimized. More particularly, it is an object of the invention to provide a system for fall prevention that is capable of accurately warning a person if a higher risk of falling occurs, which system at the same time is easy to use.

In order to achieve this object, the system according to the invention is characterized in that the system comprises a number of sensors attachable to at least one lower body segment, wherein said sensors are adapted to measure movement of said at least one lower body segment and to translate the movement into a signal, the system further comprising a control adapted to receive the signal from said respective sensors, wherein in use the control observes the signal as an actual sequence of postures of said at least one lower body segment and compares the actual sequence with a predetermined sequence of postures as a function of time, wherein the control is adapted to determine a high risk of falling when the actual sequence deviates from the predetermined sequence in a certain way.

Due to the change in sequence of postures over time in relation to a known sequence that represents a low risk of falling, the system is able to accurately detect (temporarily) higher risk of falling. This results in a dynamic way of monitoring a user during movement, for instance during walking, over a period of time. The system is able to detect a situation of imbalance of the user on time such that the user or a care provider can take precautions. For instance, when a user is not paying full attention to the walking because he is talking, listening to the radio etc., the movement of the person can provide a higher risk of falling, which is detected by the system and warns the user. It is also possible that another person, for instance a nurse, can be alerted when a higher risk of falling is indicated by the system. In that case, the nurse can accompany said user in order to prevent him from falling.

According to a further aspect of the invention, the posture of the lower body segment is determined by the position of lower body segment parts relative to each other. The lower body segment parts preferably comprise an ankle, a foot, a knee, a lower leg, an upper leg, a hip of a similar lower body segment and/or a trunk. By only monitoring the mechanical system of the leg (or of both legs), for example, determining relative coordinates of the body segment parts, i.e. the positions of those body segment parts relative to each other, a relative simple system for fall protection is provided. The risk of falling can be derived from the degree of maintaining stability, i.e. being in balance, which for instance can be inferred from the degree of bending of the knee, the degree of bending of the hip and/or the degree of bending of the ankle, such in reference to a criterion of stability in that bending, for example the usual mean or variance.

According to a further aspect of the invention, a comparison of the actual sequence of postures to the predetermined sequence of postures of the lower body segment is performed with aid of an adaptive algorithm, for example a neural network or a support vector machine. Such an algorithm enables the system to be dynamical, flexible and easy adaptable.

According to another aspect of the invention, the system is configured to monitor a muscle strength or power of muscles of the lower body segment, e.g., using EMG, and configured to use a detected muscle strength or power in the determining of the high risk of falling. Muscle strength or power relates to the balance of a user, i.e. the stability of the mechanical system of the user. Thus, detection of muscle strength or power contributes to indicating the risk of falling.

According to another aspect of the invention, the predetermined sequence of postures of the lower body segment is determined by measuring successive lower body segment postures during normal movement of the user and the amount of variation therein. By doing so, the system learns a normal sequence of postures of at least one lower body segment when a person is moving, for instance walking, with a low risk of falling. By also measuring the amount of variation in the sequence, the system learns to what extent the normal sequence is staying within the level of low risk of falling, thereby preventing to warn the user to often or without needing to.

In further elaboration of the invention, the deviation of the actual sequence of postures in relation to the predetermined sequence of postures is based on the increase or decrease in variation in the sequence as a function of time.

According to a further elaboration of the invention, the high risk of falling is determined by a deviation threshold that is estimated from a mean and the variation by classifying the actual sequence of postures. For instance, a mean of the signals is determined and the trend therein is monitored. When a deviation in the means occurs, a signal is generated to warn a user or another person. For example, when a user becomes fatigue not only a single movement is influenced. By using the deviation in the mean of the signals, the degree of fatigueness is represented in the trend in movement.

According to another aspect of the invention, the system is adapted to provide a warning signal, during walking, when the high risk of falling has been determined. Such a warning signal can be given to the user wearing the system for fall prevention, but can also be given to for instance a caretaker of the user, such as a nurse. The caretaker is then able to help the user in order decrease the high risk of falling at that time. The warning signal can be an audible signal or a visual signal, like a warning text on a display or a flashing light.

In further elaboration of the invention, the system comprises a memory for storing the sequence of postures of the at least one lower body segment. Such a memory enables the predetermined sequence of postures being dynamical by storing latest sequences in the memory and by recalibrating the adaptive algorithm occasionally, by using the sequences available in the memory at that time. Preferably, sequences in alarm situations are removed from the memory. These sequences can however be collected and used to train the algorithm to learn a category of risk patterns.

In another aspect of the invention, the adaptive algorithm is self-learning by adaptation of the predetermined sequence of postures in case of changing conditions of the user. The system first gradually learns the normal walking pattern of the user in order to be able to differentiate between a normal and a dangerous pattern. With changing conditions, because for example the user gets older and the pattern of normal walking changes, the algorithm learns that the changed patterns are the normal sequence of postures.

In further elaboration of the invention, the system is configured to monitor an angle between a lower leg and an upper leg of the user, to determine whether a high risk of falling is reached during walking of the user. Thus, not the position of the separate lower body segment parts with respect to a certain plane, for instance the horizontal plane is measured, but the position of the separate parts relative to each other.

Preferably, the sensor is one of an accelerometer, a gyroscope or a magnetometer. These sensors enable easy detection of the posture of the upper leg-lower leg system. The sensor may be miniature and/or wireless sensors, such that it is not inconvenient for the user wearing said sensors. The sensors can be adapted to continuously measure the relative posture of the lower body segment parts. It is also possible that other kinds of sensors can be used to determine the posture of the upper leg-lower leg system.

According to another embodiment of the invention, the predetermined sequence of postures can be determined by entering parameters into the control. Instead of training and tracking the actual sequences of postures of the lower body segment, it is then possible to train and track on the sequences determined by the entered parameters. The parameters can be chosen from, but is not restricted to, the group of: an amount of knee-bending over a certain time period, an average of knee-bending over a certain time period, a range of amount of knee-bending over a certain time period, a variation of the amount of knee bending over a certain time period, a step size, a left (right) knee stretching in response to right (left) knee bending.

When a person becomes fatigue, muscle strength changes and knee-bending will change. The amount of variation can increase but the person will also apply passive stability, when, e.g., the left knee bends more (because of fatigue), the left step size will reduce and the person will stretch the right leg to regain stability through that leg. This is usually unnoticed (unconscious) behaviour. Hence, an electronic indicator that detects these unconscious changes can be helpful for the user to realize his/her risk of falling is temporary increased. Changes appear as an alteration in mean value or as an alteration in the variance around that mean. Similar to fatigue, other influences can cause an increased risk of falling. For example, distraction of the user's attention to his/her walking. The increased risk for falling follows from a less stable and smooth movement pattern.

The invention further relates to a method for fall prevention for a user, using an above described system, wherein movement of at least one lower body segment is measured and translated into a signal, wherein successive signals are translated into an actual sequence of postures of said at least one lower body segment, wherein the actual sequence is compared with a predetermined sequence of postures over a certain time period, wherein a high risk of falling is being indicated when the actual sequence deviates from the predetermined sequence to a certain degree. Such a method for fall prevention provides similar advantages and effects as are mentioned with the description of the system for fall prevention.

### BRIEF DESCRIPTION OF THE DRAWINGS

The invention will be further elucidated by means of exemplary embodiments with reference to the accompanying drawings in which:

FIG. 1 shows a mechanical system of the lower body segment comprising sensors; and

FIG. 2 shows a diagram of a system according to an embodiment of the invention.

### DETAILED DESCRIPTION OF THE EMBODIMENTS

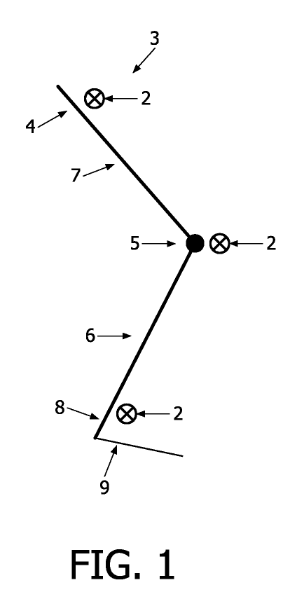

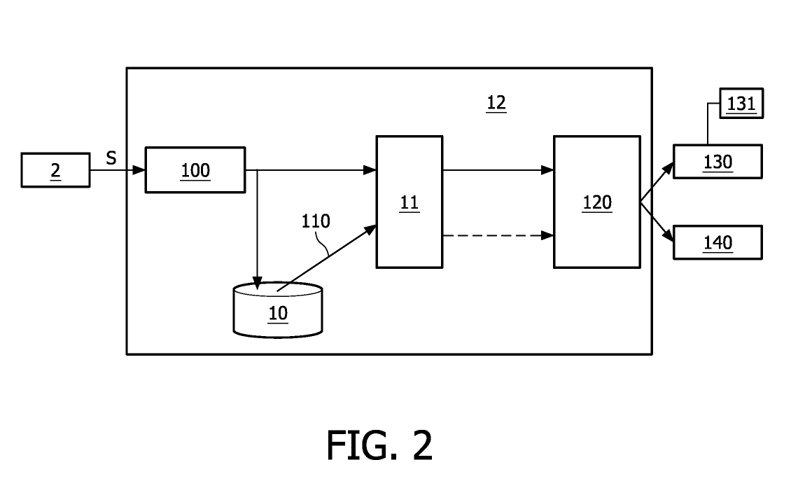

FIG. 1 illustrates a system for fall prevention for a user. A number of sensors 2 is attached to a lower body segment 3, for example a leg of a user. The sensors 2 are adapted to measure movement of the lower body segment 3 and to translate said movement into a signal S. As depicted in FIG. 2, the signal S of the sensors 2 is received by a control 12. The control 12 translates the signal into an actual sequence of postures of the lower body segment 3. The signal S is converted into an actual sequence of postures at operation 100. The actual sequence of postures is then compared by control 12 with a predetermined sequence of postures as a function of time, wherein the predetermined sequence relates to a low risk of falling or the usual risk for that user. The control 12 is further adapted to determine a high risk of falling when the actual sequence deviates from the predetermined sequence to a certain degree. The comparison of the actual sequence of postures to the predetermined sequence of postures of the lower body segment 3 is performed with aid of an adaptive algorithm 11, for example a neural network or a support vector machine.

For determining the predetermined sequence of postures, successive lower body segment 3 postures during normal movement of the user and the amount of variation therein can already have been measured. The predetermined sequence can be stored in a memory 10 of the system. The adaptive algorithm 11 can be configured with preset coefficients, in which case storage in the memory 10 and operation 110 is not required. However, better performance can be obtained when the coefficients are trained, through operation 110, from the predetermined sequences stored in the memory 10. This allows for a better comparison result with the actual pattern. Also, if the user alters his/her normal movement patterns, the algorithm 11 can adapt to those patterns through a new learning cycle 110.

More particularly, in FIG. 1 a mechanical system of the lower body segment 3 is shown. The posture of the lower body segment 3 is determined by the position of at least two lower body segment parts 6, 7 relative to each other. The lower body segment parts can be two of the following: foot 9, ankle 8, lower leg 6, knee 5, upper leg 7, hip 4, and/or trunk (not shown). Three sensors 2 are provided on respectively the ankle 8, knee 5 and hip 4 of a person in order to perform a positional measurement of that lower body segment 3. From said positions the body segment's angle can be computed. When the sensors 2 measure angular position of said lower body segment 3, it suffices to use only two sensors 2, preferable on the lower leg 6 and the upper leg 7, or on the ankle 8 and foot 9. As indicated in FIG. 1, accelerometers 2 are attached to the upper leg 7 and lower leg 6 of both legs, such that the posture of the legs can be computed as a function of time. Also additional sensors for calibration purposes can be provided (not shown). Sensors 2 can be placed on one leg or on both legs. When the user walks a trajectory, the sequence of postures of both legs can be sampled and stored in the memory 10. The sequence is used to adapt the adaptive algorithm 11.

The predetermined sequence is used during operation of the system 1 for fall prevention. The actual sequence of postures of the lower body segment 3 is monitored, during walking, and compared with the sequences that the algorithm 11 is trained with, e.g. through the sequences that are stored in the memory 10 (at operation 110). If the actual sequence of postures deviates from the predetermined sequence, i.e. the actual pattern is not recognized to match one of the patterns stored in the memory 10, the user is warned for instance with a warning signal (operation 130), for example via a loudspeaker 131 or in a different way. If the deviation is relatively small, there is low risk of falling (operation 140) and the user is not alerted. The system 1 can, instead of giving a warning signal, provide the user with an advice, for instance taking a break etc. Instead of matching the actual pattern with the stored patterns, the algorithm 11 can also compute statistical parameters such as mean and variance of the actual sequence. These numbers can be compared with those of the earlier sequences stored in the memory 10. This comparison is done in a comparator 120. If the actual mean or variance surpasses a deviation threshold relative to those from the earlier sequences, the user is warned for instance with a warning signal (operation 130), for example via a loudspeaker 131 or in a different way. If the deviation is relatively small, there is low risk of falling (operation 140) and the user is not alerted.

Adaptation of the adaptive algorithm 11 is focused on learning normal situations and developing a variation therein. A deviation threshold can be estimated form the mean and variation in classifying the normal sequences. It is assumed that an insignificant number of sequences of high-risk situations is available, therefore the adaptive algorithm 11 is adapted to learn a reliable classification of risk situations. The adaptive algorithm 11 does not classify the sequences but it returns a degree of fitting into the classification, i.e. the distance to the mean of the class. This distance is compared with the spread of learning samples in said class. It is also possible that the adaptive algorithm 11 is adapted to perform a clustering with the sequences of the postures in the memory 10 together with the actual sequence of postures. If the actual sequence is put in a different cluster than the predetermined sequences, a situation of high risk for falling is detected.

The predetermined sequences can be dynamic in the sense that they can be adapted, for instance due to a change in the user's conditions. Therefore, the latest actual sequences are stored in the memory 10 and the adaptive algorithm 11 is recalibrated once a while, using the latest actual sequences from the memory 10. Alarmed situations can be removed from the memory 10 and can be collected in order to learn the algorithm a category of risk sequences. The above-described system for fall prevention provides a simple and inexpensive way of preventing a user for falling. Furthermore, the system is very accurate and can take into account behaviour of a user that creates a higher risk of falling.

Although illustrative embodiments of the present invention have been described in greater detail with reference to the accompanying drawings, it is to be understood that the invention is not limited to these embodiments. Various changes or modifications may be effected by one skilled in the art without departing from the scope or spirit of the invention as defined in the claims.

For example, it is clear that sensors are placed on both lower body segments to determine the sequence of postures of both legs at the same time, thereby providing an accurate fall prevention system.

According to embodiments of the present invention, sensors are applied to determine sequences of body segment postures during steady state phases of movement. Particularly, the sequences of lower body segment postures (for example in combination with measurements of muscle strength or power of muscles) can provide accurate information concerning the risk of falling, since the balance of a user is (mostly) dependent on the system of hip, knee and ankle. For example, when a user is getting tired, it gets harder to normally stretch the knee (often referred to as knee buckling). Also, when it is harder for a user to stay balanced, it is associated with a larger sway (movement of the hips ). Another, often used model of balance is the inverted pendulum, taking the ankle as a pivoting point.

It is to be understood that in the present application, the term “comprising” does not exclude other elements or steps. Also, each of the terms “a” and “an” does not exclude a plurality. Any reference sign(s) in the claims shall not be construed as limiting the scope of the claims. Also, a single control, or other unit may fulfil functions of several means recited in the claims.

1. A system for fall prevention for a user, comprising a number of sensors (2) attachable to at least one lower body segment (3), wherein said sensors (2) are adapted to measure movement of said at least one lower body segment (3) and to translate the movement into a signal (S), the system further comprising a control (12) adapted to receive the signal (S) from said respective sensors (2), where in use, the control (12) observes the signal (S) as an actual sequence of postures of said at least one lower body segment (3) and compares the actual sequence with a predetermined sequence of postures as a function of time, wherein the control (12) is adapted to determine a high risk of falling when the actual sequence deviates from the predetermined sequence.

2. The system according to claim 1, wherein the posture of the lower body segment (3) is determined by the position of at least two lower body segment parts (6, 7) relative to each other.

3. The system according to claim 2, wherein the lower body segment parts comprise an ankle (8), a foot (9), a knee (5), a lower leg (6), an upper leg (7), hip (4) of a similar lower body segment (3) and/or a trunk.

4. The system according to claim 1, wherein a comparison of the actual sequence of postures to the predetermined sequence of postures of the lower body segment (3) is performed with aid of an adaptive algorithm (11).

5. The system according to claim 1, configured to monitor a muscle strength or power of muscles of the lower body segment (3), and configured to use a detected muscle strength or power in the determining of the high risk of falling.

6. The system according to claim 1, wherein the predetermined sequence of postures of the lower body segment (3) is determined by measuring successive lower body segment (3) postures during normal movement of the user and the amount of variation therein.

7. The system according to claim 6, wherein the deviation of the actual sequence of postures in relation to the predetermined sequence of postures is based on the increase or decrease in variation in the sequence as a function of time.

8. The system according to claim 6, wherein the high risk of falling is determined by a deviation threshold that is estimated from a mean and the variation by classifying the actual sequence of postures.

9. The system according to claim 1, wherein the system is adapted to provide a warning signal to the user, during walking, when the high risk of falling has been determined.

10. The system according to claim 1, wherein the system comprises a memory (10) for storing the sequence of postures of the at least one lower body segment (3).

11. The system according to claim 1, wherein the system is self-learning by adaptation of the predetermined sequence of postures in case of changing conditions of the user.

12. The system according to claim 1, wherein the predetermined sequence of postures is determined by entering parameters into the control (12) preceding using the system (1).

13. The system according to claim 12, wherein the parameters are chosen from the group of: an amount of knee-bending over a certain time period, an average of knee-bending over a certain time period, a range of amount of knee-bending over a certain time period, a variation of the amount of knee bending over a certain time period, a step size, a left (right) knee stretching in response to right (left) knee bending.

14. A method for fall prevention for a user, wherein movement of at least one lower body segment (3) is measured and translated into a signal (S), wherein successive signals (S) are translated into an actual sequence of postures of said at least one lower body segment (3), wherein the actual sequence is compared with a predetermined sequence of postures over a certain time period, wherein a high risk of falling is being indicated when the actual sequence deviates from the predetermined sequence to a certain degree.
